(19) United States
(12) Patent Application Publication
(10) Pub. No.: US 2023/0206745 A1
(43) Pub. Date: Jun. 29, 2023

(54) System for Reminding a User to Brush the User's Teeth

(71) Applicant: SONOVA AG, Staefa (CH)

(72) Inventors: Nicholas Ohs, Zürich (CH); Nadim El Guindi, Zürich (CH); Nina Stumpf, Maennedorf (CH); Niklas Ignasiak, Zürich (CH); Anna M. Parker, Basel (CH); Manuela Feilner, Egg b. Zürich (CH); Hans-Ueli Roeck, Hombrechtikon (CH)

(21) Appl. No.: 17/563,517

(22) Filed: Dec. 28, 2021

(51) Int. Cl. G08B 21/24; G01P 15/00; H04R 1/10; A61B 5/00; A61B 7/00; G08B 3/10; G06F 3/16; H04R 1/08

(52) U.S. Cl. CPC G08B 21/245; G01P 15/00; H04R 1/1016; A61B 5/7221; A61B 7/00; H04R 1/1041; A61B 5/7267; G08B 3/10; G06F 3/167; A61B 5/7257; A61B 5/6803; H04R 1/08; A61B 5/741; A61B 2562/0219; A61B 2562/0204

(57) ABSTRACT

An illustrative system includes two wearable devices configured to be worn near a jaw on opposite sides of a user's head, each device comprising: at least one microphone, at least two accelerometers, at least one loudspeaker, wherein the system is configured to process data acquired by the accelerometers and by the microphones to: detect chewing, and output an audible notification via the loudspeakers to notify the user to brush the user's teeth after chewing is no longer detected.

### BACKGROUND INFORMATION

Dental health conditions are associated with comorbidities. Elderly people may develop a tendency to neglect dental hygiene. This may have several reasons: Firstly, it may be hard for the elderly to remember applying dental hygiene on a regular basis. Secondly, even if done regularly, it can be hard for elderly people to perform dental cleaning properly, in particular in terms of time spent on cleaning, and in terms of which sector (top left, top right, bottom left, bottom right) is cleaned to what extent.

Current electric toothbrushes have inbuilt timers to remind users of the recommended cleaning time. They also have pressure sensors to avoid excessive pressure during cleaning. However, they cannot monitor which sector is cleaned nor do they announce which of the four sectors should be cleaned, which may be important for people with dementia. Furthermore, electric toothbrushes cannot remind users to clean their teeth if the user is not close to the toothbrush.

Existing reminders are limited to calendar entries that do not adapt dynamically to the real-life situation of a user, i.e. they do not react to times when the user eats.

A known solution to the above stated problem is to hire a geriatric nurse.

Another solution could be to install a camera-system loud-speaker combination in the bathroom which could monitor the tooth cleaning of the user. This has several drawbacks. First, such a system cannot remind the user to clean their teeth. Secondly, while the site could be approximately detected in the video signal, contact pressure on the teeth is not visible from the outside. Therefore, the quality of the cleaning can only be assessed in a limited fashion.

CN 113257277 A describes a tooth brushing behavior monitoring method, device and equipment, a medium and a chip system. The method can be applied to wearable equipment, wherein the wearable equipment collects audio signals and sensor data when a user brushes teeth, then the audio signals and the sensor data are recognized to obtain a recognition result, according to the recognition result, the tooth brushing behavior state of the user is determined, and when the tooth brushing state does not conform to the preset behavior specification, reminding information is sent out. According to the method, the recognition result is obtained by combining the audio signal and the sensor data, and whether the current tooth brushing behavior state conforms to the preset behavior specification or not is determined according to the recognition result, so that whether the tooth brushing behavior of the user is standard or not can be accurately recognized.

There remains a need for a solution that dynamically detects when a user should clean their teeth to remind them in due time. The solution should also monitor the cleaning procedure to ensure that the teeth are cleaned thoroughly and evenly.

### BRIEF DESCRIPTION OF THE DRAWINGS

The accompanying drawings illustrate various embodiments and are a part of the specification. The illustrated embodiments are merely examples and do not limit the scope of the disclosure. Throughout the drawings, identical or similar reference numbers designate identical or similar elements.

FIG. 1 is a schematic view of a system for detecting that a user is chewing, to remind them to brush their teeth and to monitor tooth brushing.

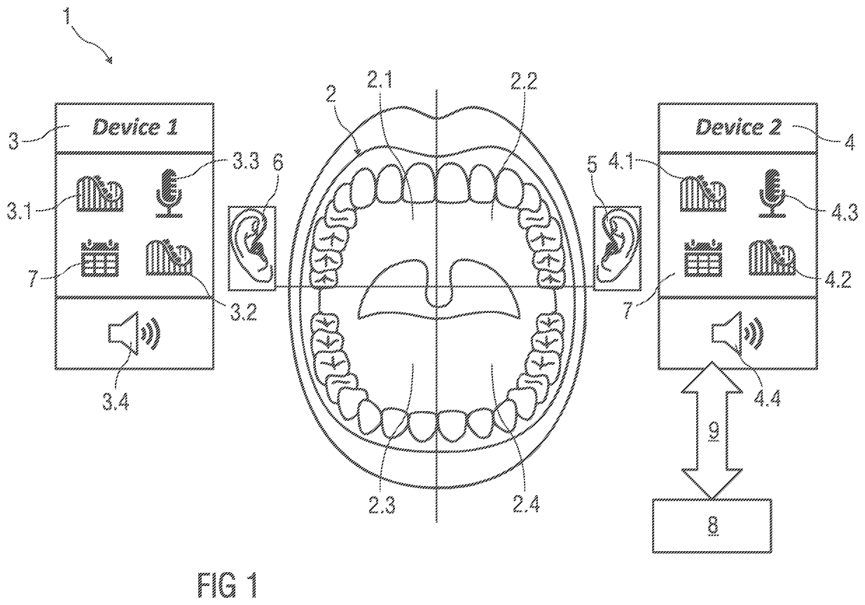

### DETAILED DESCRIPTION

Described herein is a system for detecting that a user is chewing, to remind them to brush their teeth and to monitor tooth brushing.

It is a feature of the embodiments described herein to provide a solution for the above described problem.

In some examples, a system may include two wearable devices configured to be worn near a jaw on opposite sides of a user's head, each device comprising:

at least one microphone,

at least two accelerometers,

at least one loudspeaker,

one of a calendar and access to a calendar,

wherein the system is configured to process data acquired by the accelerometers and by the microphones to:

detect chewing,

detect tooth brushing,

monitor which teeth or sector of a set of teeth is being brushed,

wherein the system is further configured to:

output an audible notification via the loudspeakers to notify the user to brush their teeth after chewing is no longer detected,

output audible notifications via the loudspeakers as to which teeth or sector of a set of teeth to brush for how much time.

In an exemplary embodiment, each device is configured as a hearing device, in particular a hearing aid, configured to be positioned at least partially in a user's ear, wherein the accelerometers of each device comprise a first accelerometer configured to be in an ear canal of the ear and a second accelerometer configured to be behind the ear.

In an exemplary embodiment, detecting chewing comprises determining periodic movement within pre-defined frequency and acceleration ranges in the data acquired by the accelerometer.

In an exemplary embodiment, determining periodic movement comprises determining a spectrum by performing a fast Fourier transform followed by selecting one or more dominant frequencies within the spectrum.

In an exemplary embodiment, a maximum acceleration is computed for each detected dominant frequency and the periodic movement is interpreted as chewing if the maximum acceleration is within pre-defined lower and upper bounds.

In an exemplary embodiment, duration of the periodic movement is analyzed to evaluate if it is within a pre-defined range indicative of chewing.

In an exemplary embodiment, the system is further configured to detect and analyze acceleration differences in the data of the first accelerometer and the second accelerometer for at least one of the devices to differentiate chewing from other types of movement.

In an exemplary embodiment, an orientation of the first accelerometer and the second accelerometer is detected in two opposite phases of the periodic movement, wherein the periodic movement is identified as not chewing if the orientation of the first accelerometer relative to the second accelerometer remains the same within a predetermined range in both phases.

In an exemplary embodiment, the system is further configured to process audio-signals detected by the one or more microphones using a trained neural network to identify false positive detection of chewing.

In an exemplary embodiment, the system is further configured to use context-data from the calendar to assess a probability that the user is eating when chewing is detected to identify false positive detection of chewing.

In an exemplary embodiment, the system is further configured to output a voice notification via the loudspeakers to remind the user to brush their teeth when a pre-determined time interval after the detected chewing has expired.

In an exemplary embodiment, the system is further configured to enter a tooth brushing monitoring mode upon one or more of the following conditions:
user input at a user interface,
vibration detection using one or more of the accelerometers,
detecting tooth brushing sounds by processing the audio-signals detected by the one or more microphones using an acoustic classifier.

In an exemplary embodiment, the system is further configured to enter a tooth brushing monitoring mode upon if one or more of the following conditions apply:
user input occurs at a user interface,
vibration is detected by one or more of the accelerometers,
tooth brushing sounds are detected by processing the audio-signals detected by the one or more microphones using an acoustic classifier.

In an exemplary embodiment, the system is further configured to locate in which sector of the set of teeth the user is currently brushing by analyzing and comparing accelerometer and microphone data of the two devices during a brushing phase.

In an exemplary embodiment, the sector where the toothbrush is currently being used is determined by taking account of an amplitude difference in the audio signals measured by the microphones of both devices, wherein the device measuring the higher amplitude is considered to be on the side where the toothbrush is currently being used.

In an exemplary embodiment, wherein the sector where the toothbrush is currently being used is determined by taking account of an amplitude difference in the accelerometer signals indicative of whether the toothbrush is currently being used on the lower or upper jaw.

In an exemplary embodiment, the system further comprises a toothbrush, wherein the toothbrush and the devices are configured to interconnect by a wireless data connection as soon as the system is in tooth brushing monitoring mode.

In an exemplary embodiment, the toothbrush is configured to determine its orientation in space and transmit this information to the devices, wherein the sector where the toothbrush is currently being used is determined taking account of the orientation.

In an exemplary embodiment, the system is further configured to verify user compliance with the requested teeth or sector of the set of teeth to be brushed and to output another audio notification in case of non-compliance.

In an exemplary embodiment, the system is further configured to analyze a vibration frequency and magnitude in the data of one of the accelerometer and a toothbrush wirelessly coupled to the devices to estimate a quality of the brushing, wherein very low frequency and/or magnitude is indicative of insufficient cleaning, wherein, another voice notification is output to notify insufficient cleaning.

The skilled person will readily understand that the features of any claim herein may be combined with the features of any other claim or any other combination of claims.

Embodiments described herein may combine chewing detection and dental hygiene monitoring, wherein the monitoring provides site-specific feedback, preferably in a form-factor that would fit into hearing devices.

The presently proposed solution is cheaper than hiring a geriatric nurse, and allows the end user to stay independent for longer.

The presently proposed solution is less complicated and potentially less expensive setup then a camera based system, in particular since hearing aids are products typically used by many elderly anyways.

Further scope of applicability of the embodiments described herein will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

In some examples, the system 1 is configured to detect if a user is chewing and to trigger a notification to brush teeth 2 in response. Furthermore, the system 1 may be configured to monitor a tooth brushing operation and to provide tooth cleaning quality feedback for the user.

The system 1 comprises two wearable devices 3, 4, each of them having one or more accelerometers 3.1, 3.2, 4.1, 4.2, one or more microphones 3.3, 4.3, and one or more loudspeakers 3.4, 4.4 configured to be acoustically coupled to an ear 5, 6 of a user so that the loudspeaker 3.4 of one of the devices 3 may be acoustically coupled to a right ear 6 of the user and the loudspeaker 4.4 of the other one of the devices 4 may be acoustically coupled to the left ear 5 of the user.

In an exemplary embodiment, the devices 3, 4, may have access to a calendar 7, e.g. via a mobile phone connection or they may comprise an internally stored calendar 7.

In an exemplary embodiment, each device 3, 4 may be configured to be placed close to one side of a jaw of a user, e.g. at or at least partially in the user's ears 5, 6.

In an exemplary embodiment, the devices 3, 4 are configured as hearing devices respectively having two accelerometers 3.1, 3.2, 4.1, 4.2, a first one 3.1, 4.1 of them configured to be in an ear canal of the respective ear 5, 6 and a second one 3.2, 4.2 of them configured to be behind the ear 5, 6 when the hearing device is correctly positioned at the user's ear 5, 6. In other embodiments, the devices 3, 4 may be headphones.

In an exemplary embodiment, in order to detect chewing, the system 1 is configured to analyze data from the accelerometers 3.1, 3.2, 4.1, 4.2 of each device 3, 4, e.g. to determine periodic movement within pre-defined frequency and acceleration ranges, The analysis may comprise determining a spectrum by performing a fast Fourier transform (FFT) followed by selecting one or more dominant frequencies within the spectrum. For each detected dominant frequency, a maximum acceleration may be computed and compared to a pre-defined acceptable lower and upper bound. Furthermore, a duration of the periodic movement, i.e. a difference in time between the first and the last detected periodic movement, may be analyzed to evaluate if it is within a pre-defined range. The duration may for example be determined by a machine learning algorithm training a neural network.

Moreover, the system 1 may be configured to detect and analyze acceleration differences between the first accelerometer 3.1, 4.1 and the second accelerometer 3.2, 4.2 of one or both devices 3, 4 to differentiate chewing from other types of movement, e.g, whole body movement such as walking. This may be achieved by comparing the orientation of the accelerometers 3.1, 3.2, 4.1, 4.2 in two opposite phases of the periodic movement, e.g. in a phase where the lower jaw is close to the upper jaw and in a phase where the lower jaw is spaced from the upper jaw. If their relative orientation towards each other remains identical within a certain range in both phases, the detected movement is not chewing.

Moreover, in order to identify false positive detection of chewing, the system 1 may be configured to process audio-signals detected by the one or more microphones 3.3, 4.3 using a trained neural network.

Moreover, in order to identify fare positive detection of chewing, the system 1 may be configured to use context-data from the calendar 7, e.g. time of day, to assess the probability that the user is eating when chewing is detected.

A resulting decision tree may detect when the user is eating. The system 1 may further be configured to output a voice notification via the loudspeakers 3.4, 4.4 to remind the user to brush their teeth 2, when a pre-determined time interval after the detected chewing has expired. The pre-determined time interval may be user set. A default range may be 1 minute to 5 minutes. Further notifications may optionally be activated with additionally configured time periods.

In contrast to only using calendar entries as in known solutions, the solution of the embodiments described herein will detect if the user actually ate anything and will help the user to remember to brush their teeth 2 even if their eating habits are irregular.

The system 1 may further be configured to enter a tooth brushing monitoring mode upon one or more of the following conditions:
user input at a user interface,
vibration detection using one or more of the accelerometers 3.1, 3.2, 4.1, 4.2,
detecting tooth brushing sounds by processing the audio-signals detected by the one or more microphones 3.3, 4.3 using an acoustic classifier.

In an exemplary embodiment, the system 1 may be configured to notify the user via audio notifications of which teeth 2 or sector 2.1, 2.2, 2.3, 2.4 of the set of teeth 2 to clean next (e.g. top left, top right, bottom left, bottom right, or similar). After a predetermined time interval, the next sector 2.1, 2.2, 2.3, 2.4 is announced via audio notifications.

Moreover, the system 1 may be configured to a) identify the type of toothbrush 8 used (e.g. electronic or manual), e.g. by analyzing sound and/or accelerometer data, and b) locate in which sector 2.1, 2.2, 2.3, 2.4 of the set of teeth 2 the user is currently cleaning by analyzing and comparing accelerometer and microphone data of the two devices 3, 4 during a cleaning phase. For example, the location where the toothbrush 8 is currently being used may be determined by taking account of an amplitude difference in the audio signal measured by the microphones 3.3, 4.3 of both devices 3, 4, right and left. The device 3, 4 measuring the higher amplitude can be considered to be on the side where the toothbrush 8 is currently being used.

Accelerometer amplitude differences can further be used to determine the difference between lower and upper jaw, due to the dampening of sound transmittance through the jaw joint, i.e. the accelerometer 3.1, 3.2, 4.1, 4.2 will detect a lower amplitude when the toothbrush 8 is being used on the lower jaw. This detection may for example likewise be achieved using a trained neural network.

In an exemplary embodiment, the system 1 may have a trained neural network implemented to identify the location of tooth brushing based on accelerometer and audio data, potentially up to the level of individual teeth.

In an exemplary embodiment, the toothbrush 8 and the devices 3, 4 may be configured to establish a wireless data connection 9 to each other, e.g. using a Bluetooth connection, in particular if the toothbrush 8 is an electronic toothbrush 8. The system 1 may be configured to establish the wireless data connection 9 between the devices 3, 4 and the toothbrush 8 as soon as they are in tooth brushing monitoring mode. The toothbrush 8 may be configured to determine its orientation in space and transmit this information to the devices 3, 4. The orientation would be indicative of the current site of cleaning. The system 1 may be configured to verify if the user is cleaning the site that has been specified by the audio notification, and output another audio notification in case of non-compliance to remind the user of the teeth 2 to be cleaned.

Moreover, the system 1 may be configured to analyze a vibration frequency and magnitude to estimate the quality of the cleaning. In an exemplary embodiment, very low frequency and/or magnitude indicate insufficient cleaning. The assessment whether a frequency and/or magnitude is very low may likewise be done by a trained neural network. If such an insufficient cleaning is detected, another voice notification may remind the user to try to increase the frequency and/or pressure, depending on whether the frequency and/or the magnitude was found to be below a set threshold. This information could again come from a wirelessly connected toothbrush 8.

Compared to known solutions which only contain a timer and check that not too much pressure is applied, the current solution is also able to detect the site of cleaning. This is particularly advantageous for the elderly and users suffering from dementia.

The analyses and method steps described herein may be performed in one or both devices 3, 4 which may have circuitry suitable to do this. The devices 3, 4 may preferably be interconnected in a wireless or wired way to facilitate operation of the system.

### LIST OF REFERENCES

1 system

2 teeth

2.1, 2.2, 2.3, 2.4 sector

3 device

3.1, 3.2 accelerometer

3.3 microphone

3.4 loudspeaker

4 device

4.1, 4.2 accelerometer

4.3 microphone

4.4 loudspeaker

5 ear, left ear

6 ear, right ear

7 calendar

8 toothbrush

9 wireless data connection

In the preceding description, various exemplary embodiments have been described with reference to the accompanying drawings. It will, however, be evident that various modifications and changes may be made thereto, and additional embodiments may be implemented, without departing from the scope of the invention as set forth in the claims that follow. For example, certain features of one embodiment described herein may be combined with or substituted for features of another embodiment described herein. The description and drawings are accordingly to be regarded in an illustrative rather than a restrictive sense.

What is claimed is:

1. A system, comprising two wearable devices configured to be worn near a jaw on opposite sides of a user's head, each device comprising:
at least one microphone,
at least two accelerometers,
at least one loudspeaker,
wherein the system is configured to process data acquired by the accelerometers and by the microphones to:
detect chewing, and
output an audible notification via the loudspeakers to notify the user to brush the user's teeth after chewing is no longer detected.

2. The system of claim 1, wherein each device is configured as a hearing device configured to be positioned at least partially in a user's ear, wherein the accelerometers of each device comprise a first accelerometer configured to be in an ear canal of the ear and a second accelerometer configured to be behind the ear.

3. The system of claim 1, wherein detecting chewing comprises determining periodic movement within pre-defined frequency and acceleration ranges in the data acquired by the accelerometer.

4. The system of claim 3, wherein determining periodic movement comprises determining a spectrum by performing a fast Fourier transform followed by selecting one or more dominant frequencies within the spectrum.

5. The system of claim 4, wherein a maximum acceleration is computed for each detected dominant frequency and the periodic movement is interpreted as chewing if the maximum acceleration is within pre-defined lower and upper bounds.

6. The system of claim 3, wherein duration of the periodic movement is analyzed to evaluate if it is within a pre-defined range indicative of chewing.

7. The system of claim 3, further configured to detect and analyze acceleration differences in the data of the first accelerometer and the second accelerometer for at least one of the devices to differentiate chewing from other types of movement.

8. The system of claim 7, wherein an orientation of the first accelerometer and the second accelerometer is detected in two opposite phases of the periodic movement, wherein the periodic movement is identified as not chewing if the orientation of the first accelerometer relative to the second accelerometer remains the same within a predetermined range in both phases.

9. The system of claim 3, further configured to process audio-signals detected by the one or more microphones using a trained neural network to identify false positive detection of chewing.

10. The system of claim 3, further configured to use context-data from a calendar to assess a probability that the user is eating when chewing is detected to identify false positive detection of chewing.

11. The system of claim 1, further configured to output a voice notification via the loudspeakers to remind the user to brush the user's teeth when a pre-determined time interval after the detected chewing has expired.

12. The system of claim 1, further configured to enter a tooth brushing monitoring mode upon one or more of the following conditions:
user input at a user interface,
vibration detection using one or more of the accelerometers,
detecting tooth brushing sounds by processing the audio-signals detected by the one or more microphones using an acoustic classifier.

13. The system of claim 1, configured to enter a tooth brushing monitoring mode upon if one or more of the following conditions apply:
user input occurs at a user interface,
vibration is detected by one or more of the accelerometers,
tooth brushing sounds are detected by processing the audio-signals detected by the one or more microphones using an acoustic classifier.

14. The system of claim 1, further configured to locate in which sector of the set of teeth the user is currently brushing by analyzing and comparing accelerometer and microphone data of the two devices during a brushing phase.

15. The system of claim 14, wherein the sector where the toothbrush is currently being used is determined by taking account of an amplitude difference in the audio signals measured by the microphones of both devices, wherein the device measuring the higher amplitude is considered to be on the side where the toothbrush is currently being used.

16. The system of claim 14, wherein the sector where the toothbrush is currently being used is determined by taking account of an amplitude difference in the accelerometer signals indicative of whether the toothbrush is currently being used on the lower or upper jaw.

17. The system of claim 1, further configured to analyze a vibration frequency and magnitude in the data of one of the accelerometer and a toothbrush wirelessly coupled to the devices to estimate a quality of the brushing, wherein very low frequency and/or magnitude is indicative of insufficient cleaning, wherein, another voice notification is output to notify insufficient cleaning.

18. The system of claim 1, wherein each device is configured to access a calendar.

19. The system of claim 1, wherein the system is further configured to process the data acquired by the accelerometers and by the microphones to detect tooth brushing and monitor which teeth or sector of a set of teeth is being brushed.

20. The system of claim 1, wherein the system is further configured to output one or more audible notifications via one or more of the loudspeakers as to which teeth or sector of a set of teeth to brush for how much time.
